(19) United States
(12) Patent Application Publication
(10) Pub. No.: US 2023/0330172 A1
(43) Pub. Date: Oct. 19, 2023

(54) COMPOSITION FOR AMELIORATING UNPLEASANT STATE CAUSED BY MENSTRUATION

(71) Applicants: NATIONAL UNIVERSITY CORPORATION KUMAMOTO UNIVERSITY, Kumamoto (JP); LINK GENOMICS, INC., Tokyo (JP); ROHTO PHARMACEUTICAL CO., LTD., Osaka (JP)

(72) Inventors: Hidetaka KATABUCHI, Kumamoto (JP); Ritsuo HONDA, Kumamoto (JP); Shinichiro NIWA, Tokyo (JP); Eri KITADAI, Tokyo (JP); Hidemi MIZUNUMA, Tokyo (JP); Harumasa ARITA, Osaka (JP)

(21) Appl. No.: 18/023,119

(22) PCT Filed: Aug. 25, 2021
(86) PCT No.: PCT/JP2021/031219

§ 371 Date: Feb. 24, 2023

(30) Foreign Application Priority Data
Aug. 25, 2020 (JP) 2020-141891

(51) Int. Cl. A61K 36/736; A61P 15/00; A61K 36/899

(52) U.S. Cl. CPC A61K 36/736; A61P 15/00; A61K 36/899

(57) ABSTRACT

The object of the present invention is to provide: a composition for ameliorating an unpleasant state caused by menstruation, which is effective against discomfort occurring in women in the period from the first menstruation to menopause, particularly strong discomfort occurring in young women, has been sufficiently experienced as a foodstuff and has an anti-EMT activity; and a functional food containing the composition. A composition for ameliorating an unpleasant state caused by menstruation is prepared, which comprises at least one component selected from the group consisting of black rice, cherry blossom, and extracts thereof.

### TECHNICAL FIELD

The present invention relates to a composition and a functional food both for ameliorating an unpleasant state caused by menstruation which has been observed in many women. More specifically, the present invention relates to a functional food which is effective for young women who tends to feel strong discomfort or anxiety although an organic impairment is not particularly observed in the regenerative system thereof.

### BACKGROUND ART

The types of an unpleasant state caused by menstruation (or menstrual discomfort) include wide variety of states such as lower abdominal pain, low back pain, headache, nausea, diarrhea, a feeling of abdominal distension, depression, tiredness and reduced appetite. As the causative disease of the unpleasant state caused by menstruation, endometriosis, uterine fibroids, uterine adenomyoma and the like are known. Many women who feel strong discomfort (or an unpleasant feeling) even though any organic impairment as mentioned above is not observed particularly.

In particular, this tendency is especially true in young women who do not experience child birth. A state where any organic impairment other than the discomfort is not particularly observed is defined as “functional dysmenorrhea” (Non-Patent Document 1). This state is considered as presymptomatic disease, because a causative disease for the state is not particularly observed.

It has been reported that there are 8 million women who have dysmenorrhea including functional dysmenorrhea and organic dysmenorrhea in Japan in 2012. According to the estimation by Ministry of Economy, Trade and Industry in 2019, the work loss due to menstruation-associated clinical conditions in women exceeds 490 billion yen per year (Non-Patent Document 2). Therefore, it is not too much to say that the unpleasant state is a serious social problem as well as serious discomfort associated with female menstruation.

Meanwhile, for organic dysmenorrhea, a therapy method depending on the specific organic impairments is relatively clear. In contrast, for functional dysmenorrhea, NSAIDs, which can alleviate pain that is a typical unpleasant feeling, have been primarily taken. NSAIDs are analgesic drugs that are ingested when a pain is felt. Therefore, as a matter of course, NSAIDs cannot improve into a physical constitution where a person does not feel the pain any more. Trauma caused by the monthly events of the visit of this pain is believed to be a cause of premenstrual syndrome (PMS) or premenstrual dyspholic disorder (PMDD) that is one of psychosomatic diseases. It is difficult to ameliorate PMS or PMDD by the ingestion of NSAIDs that are drugs to be taken after the pain is felt. When the pain is too strong, it is possible to employ a treatment method in which a menstrual period that is the cause of the pain is caused to stop with a female sex hormone preparation. In addition, a therapy is also employed, in which a small quantity of a female sex hormone is administered to inhibit the maturation of ovarian follicles or the growth of an endometrium, thereby relieving menstrual pain. However, according to a questionnaire survey made on dysmenorrhea patients, the use result of female sex hormone preparations is as extremely low as 7.5% due to psychological difficulty in or adverse side effects of the forcible alternation of natural incretion of an original hormone (Non-Patent Document 3).

Furthermore, in recent years, women's advancement into society has been remarkable, and as a result, delayed marriage has become a social trend. Dysmenorrhea can occur in a period from the first menstruation to menopause. It is believed that the symptom in functional dysmenorrhea is severe in young women who have not have experience child birth. Accordingly, the social problem that the work loss due to dysmenorrhea increasingly becomes more serious because of delayed marriage. In these situations, the development of a radical treatment that can solve the serious afflictions in women and the social loss as mentioned above has been keenly demanded.

In recent years, on the other hand, the mechanism of dysmenorrhea has been made clear. The underlying mechanism of both of functional dysmenorrhea and organic dysmenorrhea is believed to be the transition of endometrial epithelial cells (i.e., epithelial-mesenchymal transition (also abbreviated as “EMT”, hereinafter)) (Patent Documents 1 and 2). When tranilast, which is a drug that has been confirmed to have an anti-EMT activity actually, was administered to dysmenorrhea patients, a significant level of improvement was observed in NRS scores with respect to pain during a menstrual period (Patent Documents 1 and 2). Accordingly, it has been keenly demanded to improve into a physical constitution that is not rarely affected by discomfort associated with menstruation by ingesting a component that has an anti-EMT activity, has little adverse side effects and is safe.

### PRIOR ART DOCUMENTS

### Patent Documents

Patent Document 1: Japanese Patent No. 4709328
Patent Document 2: Japanese Patent No. 5819599

### Non Patent Documents

Non-Patent Document 1: Guideline for obstetrical and gynecological practice in Japan—Gynecological outpatient edition, 2017
Non-Patent Document 2: the Ministry of Economy, “Approach to women's health in health management”, the Ministry of Economy, Trade and Industry, 2019
Non-Patent Document 3: News release published by Jun. 1, 2018, “Study on awareness and fact-finding survey about endometriosis and menstruation management”, Bayer Pharma Japan.

### SUMMARY OF THE INVENTION

### Problems to be Solved by the Invention

The object of the present invention is to provide: a composition for ameliorating an unpleasant state caused by menstruation, which is effective against discomfort occurring in women in the period from the first menstruation to menopause, particularly strong discomfort occurring in young women, has been sufficiently experienced as a foodstuff and has an anti-EMT activity; and a functional food containing the composition.

### Means for Solving the Problems

In order to solve the above-mentioned problem, extensive and intensive studies have been made. As a result, it is found that a specific plant-derived component has a significant anti-EMT activity and is effective for the amelioration of an unpleasant state associated with menstruation. This finding leads to the accomplishment of the present invention.

The present invention provides the following composition.

Item 1.

A composition for ameliorating an unpleasant state caused by menstruation, the composition comprising at least one component selected from the group consisting of black rice, cherry blossom and extracts thereof.

Item 2.

The composition according to item 1, in which the unpleasant state caused by menstruation is an unpleasant state that occurs during a menstrual period or before the menstrual period.

Item 3.

The composition according to item 1 or 2, in which the unpleasant state caused by menstruation is at least one state selected from the group consisting of lower abdominal pain, low back pain, a feeling of abdominal distension, headache, nausea, anemia, diarrhea, tiredness, depression, emotional instability, poor concentration, sleepiness, engorgement of breast (breast tenderness), swelling, rough skin, reduced appetite, decline in efficiency, body weight gain and an incapacitating state.

Item 4.

The composition according to any one of items 1 to 3, in which the black rice extract is an extract of a part of black rice which contains at least one component selected from the group consisting of a hull, an aleurone layer, a pericarp, a germ and an endosperm of black rice.

Item 5.

The composition according to any one of items 1 to 3, in which the cherry blossom extract is an extract of cherry blossom petals.

Item 6.

The composition according to any one of items 1 to 5, in which the composition is an oral preparation.

### Effect of the Invention

The composition according to the present invention comprises a component that has been sufficiently experienced as a foodstuff and has an anti-EMT activity, and therefore the composition can exhibit an effective ameliorating effect on discomfort associated with menstruation, particularly discomfort that young women often feel.

### BRIEF DESCRIPTION OF THE DRAWINGS

FIG. 1 is a graph showing the result of the evaluation of an EMT inhibition activity (percentage of inhibition) by a black rice extract in Test Example 1.

FIG. 2 is a graph showing the result of the evaluation of an EMT inhibition activity (percentage of inhibition) by a cherry blossom extract in Test Example 1.

### MODE FOR CARRYING OUT THE INVENTION

The composition according to the present invention comprises a component having an anti-EMT activity as an active ingredient, and is therefore expected to contribute to the essential amelioration of discomfort associated with menstruation, of which the mechanism has been increasingly elucidated in recent years.

The active ingredient as used herein is not particularly limited, as long as the ingredient has been sufficiently experienced as a foodstuff and has an anti-EMT activity. The active ingredient is preferably a material that has been experienced as a foodstuff in the field of foods and beverages and/or a component derived from the material, but is not limited to these components.

### [Component Having Anti-EMT Activity]

In the present disclosure, at least one component selected from the group consisting of black rice, cherry blossom, and extracts thereof is now discovered as a component having an anti-EMT activity.

### (Black Rice and Extract Thereof)

Black rice is a variety that a purplish black pigment is contained in at least one of a hull or a pericarp of an unpolished rice, and has black appearance. As the variety of black rice, “Shikokuen”, “Kurodaen”, “Shonan black rice (registered tradename), “Kokushi”, “kohokushi black rice” and the like are known. As the black rice, either one of brown rice (an unpolished rice) or a polished white rice produced by milling brown rice may be used. From the viewpoint of significantly achieving the effect of the present invention, it is preferred to use at least one selected from the group consisting of a hull or a pericarp of brown rice and a germ as the black rice.

With respect to the black rice extract, the extraction method is not limited as long as the effect of the present invention can be achieved. One example of the black rice extract is a liquid extract produced by immersing and extracting the black rice or a pulverized product thereof in and from water and/or an organic solvent and then filtrating off a residue from the resultant solution, a product produced by removing the solvent from the liquid extract or a fine powder of the product, or a product produced by dissolving, dispersing or diluting the liquid extract or a solvent-removed product thereof using a proper solvent. As the black rice extract, a commercially available product may be used. As another example of the extraction method, the black rice extract can be extracted after subjecting the black rice to a processing procedure such as a steaming procedure.

The black rice extract may be a crude extract that is extracted from the black rice, a further refined product of the crude extract, a concentrated product of the crude extract, or a synthetically produced product. As the black rice extract, a commercially available product may be used. The method for producing the extract is not particularly limited, and a common extraction method, a common purification method, a common concentration method, a common synthesis method, a common dry-mode powderization method or the like may be employed.

The extraction solvent for the black rice extract is not limited, as long as the effect of the present invention can be achieved. Examples of the extraction solvent include: a lower alcohol having 1 to 4 carbon atoms, such as methanol, ethanol, propanol, isopropyl alcohol and butanol; a polyhydric alcohol such as glycerin, propylene glycol and polyethylene glycol; a polar solvent such as water, acetone, ethyl acetate and methyl acetate; and a non-polar solvent such as hexane and pyridine. These extraction solvents may be used singly, or a mixed solvent of arbitrary two or more of these extraction solvents may be used. Among these extraction solvents, water, ethanol, 1,3-butylene glycol, propylene glycol, or a mixed solution of these solvents is preferred, and a mixed solution of water and ethanol (i.e., hydrous ethanol) is more preferred.

Examples of the commercially available product of the black rice extract to be used include, but are not limited to, black rice extract-P (manufacturer: Oryza Oil & Fat Chemical Co., Ltd.), a mixed ancient rice extract (manufacturer: Saticine Medical Co., Ltd.), and a black rice extract (manufacturer: ACCESS-ONE Co. Ltd.).

The type of the black rice extract is not limited as long as the effect of the present invention can be achieved. For example, the black rice extract has a polyphenol content of preferably 3% by mass or more, more preferably 5% by mass or more, still more preferably 10% by mass or more, particularly preferably 12% by mass or more, most preferably 15% by mass or more. The polyphenol content can be measured by, for example but not limited to, Folin-Denis method.

### (Cherry Blossom and Extract Thereof)

The term “cherry blossom” as used herein is a general term for plants belonging to the family Rasaceae, the genus Cerasus, excluding Japanese plum (Ume), peach, apricot and the like, and generally refers to a plant belonging to Subgen. Cerasus.

The type of the cherry blossom is not particularly limited. For example, cherry blossom belonging to a Japanese mountain cherry (jamazakura) group, a rose bud cherry (edohigan cherry) group, a Japanese dwarf cherry (mamezakura) group, a clove cherry (chojizakura) group, a Miyama cherry group, and a Chinese sour cherry group can be used. The cherry blossom is not limited to these groups, and other types of cherry blossom may be used. Specific examples of the cherry blossom to be used include, but are not limited to: Japanese cherry (e.g., Prunus cerasus), Prunus avium, Prunus campanulata, Prunus cerasus), Prunus jamasakura, Prunus leveilleana, Prunus pendula, Prunus pseudocerasus, Prunus speciosa, Prunus verecunda, Prunus x mochizukiana, Prunus x yedoensis and Prunus lannesiana.

The type of the cherry blossom is not limited, and is preferably Prunus lannesiana, from the viewpoint of achieving the effect of the present invention significantly.

The part of the cherry blossom to be used is not particularly limited, and examples of the part include a leaf, a stem, a shaft, a petal, a root and a fruit. Among these parts, a petal or a leaf is preferably used, and a petal is more preferably used.

With respect to the cherry blossom extract, the extraction method is not limited, as long as the effect of the present invention can be achieved. Examples of the cherry blossom extract include: a liquid extract which is produced by extracting the cherry blossom by immersing the cherry blossom or a pulverized product thereof in water and/or and organic solvent and then filtrating off a residue from the resultant product; a product which is produced by removing the solvent from the liquid extract (i.e., a solvent-removed product); a fine powder of each of these products; and a product which is produced by, for example, dissolving, dispersing or diluting the liquid extract or the solvent-removed product in or with a proper solvent. Alternatively, a commercially available product may also be used. As another example of the extraction method, the cherry blossom extract can also be extracted after subjecting the cherry blossom to a processing procedure such as a steaming procedure.

The cherry blossom extract may be a crude extract that is extracted from the cherry blossom, a further refined product of the crude extract, a concentrated product of the crude extract, or a synthetically produced product. As the cherry blossom extract, a commercially available product may also be used. The method for producing the extract is not particularly limited, and a common extraction method, a common purification method, a common concentration method, a common synthesis method, a common dry-mode powderization method or the like may be employed.

The extraction solvent for the cherry blossom extract is not limited, as long as the effect of the present invention can be achieved. Examples of the extraction solvent include: a lower alcohol having 1 to 4 carbon atoms, such as methanol, ethanol, propanol, isopropyl alcohol and butanol; a polyhydric alcohol such as glycerin, propylene glycol and polyethylene glycol; a polar solvent such as water, acetone, ethyl acetate and methyl acetate; and a non-polar solvent such as hexane and pyridine. These extraction solvents may be used singly, or a mixed solvent of arbitrary two or more of these extraction solvents may be used. Among these extraction solvents, water, ethanol, 1,3-butylene glycol, propylene glycol, or a mixed solution of these solvents is preferred, and a mixed solution of water and ethanol (i.e., hydrous ethanol) is more preferred.

As the commercially available product of the cherry blossom extract, cherry blossom petal extract-P (manufacturer: Oryza Oil & Fat Chemical Co., Ltd.), a cherry blossom petal syrup (manufacturer: Oryza Oil & Fat Chemical Co., Ltd.) and the like can be used, but the commercially available product is not limited to these products.

The cherry blossom extract is not limited as long as the effect of the present invention can be achieved. For example, the cherry blossom extract has a caffeoyl glucose content of preferably 1% by mass or more, more preferably 1.5% by mass or more, still more preferably 2% by mass or more. The content of caffeoyl glucose can be measured by a known method. For example, the content of caffeoyl glucose can be measured by, but not limited to, HPLC.

The cherry blossom extract is not limited as long as the effect of the present invention can be achieved. For example, the cherry blossom extract has a quercetun glucoside content of preferably 0.01% by mass or more, more preferably 0.03% by mass or more, still more preferably 0.05% by mass or more. The quercetun glucoside content can be measured by a known method. For example, the quercetun glucoside content can be measured by, but not limited to, HPLC.

In the composition according to the present invention, the total content of the component having an anti-EMT activity is determined appropriately depending on the type of the component, the type or content of other blend component, the dosage form of the preparation, the type of usage and the like. For example, for the production of a solid preparation, the total content of the component having an anti-EMT activity is, but not limited to, preferably 0.001% by mass or more, more preferably 0.005% by mass or more, still more preferably 0.01% by mass or more, particularly preferably 0.05% by mass or more, furthermore particularly preferably 0.1% by mass or more, most preferably 0.5% by mass or more, relative to the whole mass of the composition. For the production of a solid preparation, the total content of the component having an anti-EMT activity is preferably 95% by mass or less, more preferably 90% by mass or less, particularly preferably 85% by weight or less, most preferably 80% by mass or less, relative to the whole mass of the composition. For the production of a solid preparation, the total content of the component having an anti-EMT activity is preferably 0.001 to 95% by mass, more preferably 0.005 to 90% by mass, still more preferably 0.01 to 85% by mass, particularly preferably 0.05 to 80% by mass, relative to the whole mass of the composition. In the present disclosure, when a plant-derived extract is used, the content of the extract is determined in terms of a dry solid content.

In the composition according to the present invention, the total content of the component having an anti-EMT activity is determined appropriately depending on the type of the component, the type or content of other blend component, the dosage form of the preparation, the type of usage and the like. For example, for the production of a liquid preparation, the total content of the component having an anti-EMT activity is preferably 0.0001% by mass or more, more preferably 0.0005% by mass or more, still more preferably 0.001% by mass or more, particularly preferably 0.005% by mass or more, furthermore particularly preferably 0.01% by mass or more, most preferably 0.05% by mass or more, relative to the whole mass of the composition. For the production of a liquid preparation, the total content of the component having an anti-EMT activity is preferably 25% by mass or less, more preferably 20% by mass or less, particularly preferably 15% by weight or less, most preferably 10% by mass or less, relative to the whole mass of the composition. For the production of a liquid preparation, the total content of the component having an anti-EMT activity is preferably 0.0001 to 25% by mass, more preferably 0.0005 to 20% by mass, still more preferably 0.001 to 15% by mass, particularly preferably 0.005 to 10% by mass, relative to the whole mass of the composition. In the present disclosure, when a plant-derived extract is used, the content of the extract is determined in terms of a dry solid content.

In a preferred aspect, when the black rice and/or an extract thereof is contained as the component having an anti-EMT activity, for the production of a solid preparation, the content of the black rice and/or an extract thereof is, but not limited to, preferably 0.001 to 95% by mass, more preferably 0.005 to 90% by mass, still more preferably 0.01 to 85% by mass, particularly preferably 0.05 to 80% by mass, in terms of a dry solid content relative to the whole mass of the composition. For the production of a liquid preparation, the content of the black rice and/or an extract thereof is preferably 0.0001 to 25% by mass, more preferably 0.0005 to 20% by mass, still more preferably 0.001 to 15% by mass, particularly preferably 0.005 to 10% by mass, in terms of a dry solid content relative to the whole mass of the composition.

In a preferred aspect, when the cherry blossom and/or an extract thereof is contained as the component having an anti-EMT activity, for the production of a solid preparation, the content of the cherry blossom and/or an extract thereof is, but limited to, preferably 0.001 to 95% by mass, more preferably 0.005 to 90% by mass, still more preferably 0.01 to 85% by mass, particularly preferably 0.05 to 80% by mass, in terms of a dry solid content relative to the whole mass of the composition. For the production of a liquid preparation, the content of the cherry blossom and/or an extract thereof is preferably 0.0001 to 25% by mass, more preferably 0.0005 to 20% by mass, still more preferably 0.001 to 15% by mass, particularly preferably 0.005 to 10% by mass, in terms of a dry solid content relative to the whole mass of the composition.

### [Applications]

The composition according to the present invention comprises a component having an anti-EMT activity as an active ingredient, and is expected to contribute to the radical amelioration of discomfort associated with menstruation of which the mechanism has been increasingly made clear in recent years.

As mentioned above, the mechanism of dysmenorrhea has been increasingly made clear in recent years. Dysmenorrhea is classified into functional (primary) dysmenorrhea and organic (secondary) dysmenorrhea.

Functional (primary) dysmenorrhea is a disease in which no organic disorder is observed in the pelvis, and 90% or more of the cases of dysmenorrhea is functional (primary) dysmenorrhea. The mechanisms of the onset of both of functional dysmenorrhea and organic dysmenorrhea are not made clear sufficiently, and therefore an effective therapeutic drug for these diseases is not discovered yet. As described in Patent Documents 1 and 2, an endometrial epithelial tissue in a normal person (a non-dysmenorrhea patient) is naturally transformed from a mesenchymal phenotype to an endothelial phenotype in a phase transition period from a low temperature phase to a high temperature phase in the basal body temperature curve before ovulation. In contrast, in a dysmenorrhea patient, it is found that the change in the phenotype is protracted and a mesenchymal-like strong phenotype is maintained even in the high temperature phase.

A searching for a drug that can normalize this transition change, in other words, can change the mesenchymal-like phenotype into an epithelial-like phenotype has been made, and it is found that tranilast has an effect to cause the transition from a mesenchymal-like phenotype of an endometrial epithelial cell into an epithelial-like phenotype (i.e., an anti-EMT activity). When tranilast is administered to a plurality of dysmenorrhea patients for a certain period to verify about the effect of tranilast having the anti-EMT activity, an obvious dysmenorrhea-ameliorating effect is observed in both of functional (primary) dysmenorrhea patients and organic (secondary) dysmenorrhea patients.

From the findings described in Patent Documents 1 and 2, it is found that a component having an anti-EMT activity can maintain the change in the phenotype in an endometrial epithelial tissue in a normal state, can prevent the development of an unpleasant state associated with menstruation and can ameliorate the unpleasant state caused by menstruation.

As the unpleasant state caused by menstruation, at least one physical or mental state selected from the group consisting of the following states can be mentioned: lower abdominal pain, low back pain, a feeling of abdominal distension, headache, nausea, anemia, diarrhea, tiredness (fatigue), depression, emotional instability, poor concentration, sleepiness, engorgement of breast (breast tenderness), swelling, rough skin, reduced appetite, decline in efficiency, body weight gain and an incapacitating state. In the present disclosure, these unpleasant states can also be referred to as dysphoria occurring before the menstrual period or during the menstrual period. It is assumed that, in an endometrial epithelial tissue, a physical condition as mentioned above is developed and, as a result, a physical or mental state as mentioned above is induced when the transition of the phenotype does not occur normally and is protracted.

Examples of the state that interferes with daily life include mental and physical difficulties including the decrease in physical activity efficiency on housework or business tasks, the inability to perform housework or business tasks, the decrease in hours for active movement, the loss of a life rhythm, a feeling of restlessness, poor concentration, the diminished volition for action, a sleepoholic symptom, inertia, mental anxiety and depression.

In the present disclosure, the wording “the onset of an unpleasant state caused by menstruation is prevented and ameliorated” means that an unpleasant state caused by menstruation as exemplified above is prevented, is alleviated, or is not felt severe.

Examples of the menstruation-associated symptom include unpleasant states associated with pain (lower abdominal pain, fatigability, stiffness of shoulder, headache, pain/listlessness in lower back), dull and tightening discomfort in a lower abdominal region, concentration (poor concentration, insomnia, increase in mistakes), behavior change (tendency to stay stuck at home, terrible sleepiness, loss of patience in business tasks), retention of water (rough skin, formation of pimples, swelling, body weight gain, engorgement of breast), emotional instability (feeling of loneliness or anxiety, depression, uneven temper, restlessness, tendency to feel a frustrated or touchy feeling) and the like.

The unpleasant state caused by menstruation as exemplified above often occurs during the menstrual period (in a menstrual period) as well as before the menstrual period. The physical or mental state as mentioned above may often occur slightly about 1 to 2 weeks before the menstrual period. When the above-mentioned physical or mental state occurs too strongly to interfere with the daily life, the state is called “premenstrual syndrome (PMS)”. When the composition according to the present invention is used, it becomes possible to ameliorate, for example but limited to, the physical and/or mental unpleasant state that occurs 1 to 2 weeks before the menstrual period.

Persons who suffer from particularly severe menstruation are often affected by mental stresses such as emotional depression, demotivation, poor concentration or the like associated with the visiting of menstruation, about 1 to 2 weeks before the menstrual period. When the mental stress occurring about 1 to 2 weeks before the menstrual period is too strong to interfere with the daily life, the state is called “premenstrual dysphonic disorder (PMDD)”. When the composition according to the present invention is used, it becomes possible to ameliorate, for example but limited to, the mental stress that occurs 1 to 2 weeks before the menstrual period.

The composition according to the present invention can be added to or mixed with a food or beverage. Alternatively, the composition according to the present invention can also be used without any modification as a food or beverage. Alternatively, the composition according to the present invention may be added to or mixed with a food or beverage that is claimed as “functional” in the wordings relating to the amelioration of an unpleasant state caused by menstruation, i.e., a functional food such as a health food, a food with functional claims, a food for patients and a food for specified health uses, for use.

As the method for evaluating an unpleasant state caused by menstruation, Menstrual distress questionnaire (MDQ) score (Moos, R. H, Journal of Psychosomatic Medicine, 30:853-867, 1968) or the like can be employed.

In the MDQ score, the evaluation factors are classified into factors associated with physical clinical conditions and factors associated with mental clinical conditions. The factors associated with physical clinical conditions include (1) a pain-related factor (stiffness of neck or shoulder, pain in muscles, headache, lower abdominal pain, lower back pain, fatigability, physical pain), (2) an autonomic nerve-related factor (dizziness, lightheaded feeling, sweating, nausea, face flushing), (3) a water retention-related factor (body weight gain, rough skin, formation of pimples, engorgement of breast, edema in any one of an abdominal region, breast and feet), and (4) a control-related factor (labored breathing, feeling of chest tightening, tinnitus, limb numbness, unfocused vision, blurred vision).

The factors associated with mental clinical conditions include (1) a concentration-related factor (insomnia, tendency to become forgetful, inability to organize thoughts, inability to judge correctly, poor concentration, distracting, increase in mistakes, awkward movement), (2) a behavior change-related factor (loss of patience in jobs, napping, inability to get out of bed, feeling to want to avoid socializing, tendency to get lazy, decrease in work efficiency), (3) a negative emotion-related factor (feeling like crying about little things, loneliness, anxiety, restlessness, frustrated or touchy feeling, uneven temper and tendency to be upset, depressed feeling, tendency to get nervous), and (4) an uplifting feeling-related factor (feeling of kindness, feeling of honesty, excitement, feeling to be filled with satisfaction, feeling to become physically active).

As the method for evaluating pain during the menstrual period, (1) the grading of a subjective symptom and (2) the medical interview about an objective symptom and the like prescribed in the guideline of Japanese Society for Palliative Medicine can be employed, for example.

With respect to item (1) that relates to the guideline in “Japanese Society for Palliative Medicine” as mentioned above, the strength of pain experienced by a recipient (subjective symptom) is rated into 11 grades ranging from grade 0 (no pain is experienced) to grade 10 (worst pain imaginable is experienced so as to take a day off from work (or stay in bed) due to pain in the lower abdomen or the lower back), and is scored in accordance with “Numerical Rating Scale (NRS)”. With respect to item (2) mentioned above, it is evaluated about to what extent the daily life of a recipient is disturbed by the pain by a monthly medical interview by a physician.

For example, but not limited thereto, when a component having an anti-EMT activity is ingested by a subject and improvement in at least one evaluation item among the above-mentioned items is observed in the subject, it is concluded that the component is effective for the evaluation item.

Alternatively, for the evaluation about an unpleasant state caused by menstruation, it is also possible to record the daily state using, for example but not limited to, “PMS Memory” (published by Japan Family Planning Association). PMS Memory has been used for the diagnosis of PMS as well as the recording of the physical condition of a woman. For example, when PMS Memory is used, it is possible to record date, a menstrual period, a menstrual blood loss, a basal body weight, a basal body temperature, a clinical condition and the severity thereof, an event and the like.

In addition to the above-mentioned items, a measure for evaluating QOL or the like can also be employed as a means for evaluating an unpleasant state caused by menstruation. Examples of the measure of this type include SF-36, SF-12v2 and SF-8 that are shortened versions of SF-36, and EQ-5D-5L which are included in the measures for HR-QOL (Health-related QOL).

SF-36 is a scoring method such that evaluation is made by responding to questions about the following 8 suborder measures (subitems) and then scoring the results: (1) physical functioning, (2) daily role limitations (due to physical problems), (3) physical pain, (4) general health, (5) energy, (6) social functioning, (7) daily role limitations (due to emotional problems), and (8) emotional well-being.

EQ-5D-5L is a questionnaire developed by EuroQOL Group, in which a respondent is asked to response to five items, i.e., “Mobility”, “Self-care”, “Usual activities”, “Pain/discomfort”, and “Anxiety/depression”, on a 5-point evaluation scale.

### [Dosage Form]

Specifically, each of the health food, the food with functional claims, the food for patients and the food for specified health uses can be used in any one of various dosage forms including a solid preparation (e.g., tablets, granules, fine granules, a powder, capsules, chewable tablets), a liquid preparation (e.g., a drink, a syrup, a suspension), and a liquid meal. Each of the foods in these dosage forms can be produced in the same manner as for the known pharmaceutical preparations. For example, the food can be produced by mixing the active ingredient with a carrier that is acceptable for foods, e.g., a proper excipient, and then subjecting the mixture to a conventional means.

For example, the tablets may be produced by mixing a powdery active ingredient with a pharmaceutically acceptable carrier (e.g., an excipient) and then compression-molding the resultant mixture, and confectionary tablets such as candy, may be produced by a mold-injection method. The tablets may be subjected to sugar coating to produce sugar-coated tablets. The tablets may be single-layer tablets or multi-layer tablets such as double-layer tablets.

A powdery or granular preparation such as granules may be prepared by any one of various granulation methods (e.g., an extrusion granulation method, a milling granulation method, a dry compaction granulation method, a fluidized bed granulation method, a tumbling granulation method, a high-speed agitating granulation), and the tablets may be prepared by a proper combination of the above-mentioned granulation methods and tableting methods (e.g., a wet tableting method, a direct tableting method) and the like.

The capsules can be prepared by filling a powdery/granular preparation (e.g., a powder, granules) in capsules (e.g., soft or hard capsules) by a conventional method.

The liquid preparation can be prepared by dissolving or dispersion various components in a water-based medium that serves as a carrier component (e.g., purified water, ethanol-containing purified water), then optionally filtering or sterilizing the resultant solution, then filling the solution in a specific container, and then sterilizing the resultant product. The dosage form of the solid preparation according to the present invention is preferably capsules or tablets, more preferably soft capsules.

The soft capsules are preferred by a user, because the soft capsules have smooth surfaces and therefore are easy to be swallowed. Examples of the common soft capsule production method include a flat plate method, a rotary method, and a seamless method.

In the production by a rotary (punching) method, a flowing filling material is sandwiched by sheet-like capsule coating films and the resultant product is shaped into capsules through holes arranged in a rotating cylindrical mold. In the production by a seamless (dropping) method, a capsule coating film composition and a filling material are ejected simultaneously through concentric multi-nozzles to form seamless capsules.

The base material for a coating film for a soft capsule preparation is not particularly limited, and starch, pullulan, cellulose, polyvinyl alcohol, gelatin, succinylated gelatin or the like can be used as the base material. Among these materials, starch, gelatin and succinylated gelatin are preferred, and gelatin and succinylated gelatin are more preferred. These materials may be used singly, or two or more of them may be used in combination.

It is also possible to produce a food composition as a liquid beverage (e.g., a soup, a juice, a fruit beverage, milk, a milk beverage, a whey beverage, a lactobacillus beverage, a tea beverage, a black tea including a herb tea, an alcoholic beverage, a coffee beverage, a carbonated beverage, a refreshing beverage, a water beverage, a cocoa beverage, a jelly-like beverage, a sports drink, and a dieting beverage), a semisolid food (e.g., a pudding and a yogurt product), a noodle, a confectionery, and a spread.

The food composition may be blended with various food additives. Examples of the food additive include an antioxidant agent, a coloring agent, a flavoring agent, a seasoning agent, a sweetening agent, an acidulant, a pH modifier, a quality stabilizer, and a preservative agent.

When a pharmaceutical composition according to the present invention is prepared, a preparation comprising the component having an anti-EMT activity that serves as an active ingredient and preferably a pharmaceutically acceptable carrier is prepared. The term “pharmaceutically acceptable carrier” as used herein generally refers to, for example, an inert, non-toxic solid or liquid bulking, diluting or capsulating material that is not reactive with the active ingredient. Examples of the pharmaceutically acceptable carrier include water, a polyol, a proper mixture thereof, a solvent including a vegetable oil, and a dispersion medium.

The pharmaceutical composition can be administered orally or parenterally to, for example, the oral cavity, the digestive tract or the nasal cavity. Examples of the preparation for oral administration include a solid preparation (e.g., tablets, granules, fine granules, a powder, capsules, chewable tablets) and a liquid preparation (e.g., a syrup, a suspension, an inhalant). Examples of the preparation for parenteral administration include an intravenous drip, nasal drops, and an injection.

The pharmaceutical composition may further comprise additives that have been used conventionally in the medical field. Examples of the additive include an excipient, a binder, a disintegrating agent, a lubricant and a corrigent. The additives may be used appropriately as required. For the sustained release for the development of a long-acting effect, the pharmaceutical composition may be coated with a known retarding agent or the like. In the pharmaceutical composition, other additives or drugs (e.g., an antiacid agent, a gastric mucosal protective agent) may be added.

The pharmaceutical composition can be applied in the form of an oral composition, an internal composition or the like. The pharmaceutical composition may be used for therapeutic purposes or non-therapeutic purposes.

The amount of intake or administration of the component having an anti-EMT activity per day for an adult person through an oral route may be determined appropriately depending on the physical state, body weight, sex or age of an individual, the activity of the material, the route employed for the ingestion or administration, the schedule for the ingestion or administration, dosage form, or other factor.

The amount of intake or administration of the black rice or an extract thereof per day for an adult person through an oral route is, for example, preferably 0.001 mg/kg (body weight)/day or more, more preferably 0.005 mg/kg (body weight)/day or more, still more preferably 0.01 mg/kg (body weight)/day or more, particularly preferably 0.05 mg/kg (body weight)/day or more, most preferably 0.1 mg/kg (body weight)/day or more. The amount of intake or administration of the black rice or an extract thereof per day for an adult person through an oral route is, for example, preferably 2000 mg/kg (body weight)/day or less, more preferably 1500 mg/kg (body weight)/day or less, still more preferably 1000 mg/kg (body weight)/day or less, particularly preferably 800 mg/kg (body weight)/day or less, most preferably 500 mg/kg (body weight)/day or less. The amount of intake or administration of the black rice or an extract thereof per day for an adult person through an oral route is, for example, preferably 0.001 to 2000 mg/kg (body weight)/day, more preferably 0.005 to 1500 mg/kg (body weight)/day, still more preferably 0.01 to 1000 mg/kg (body weight)/day, particularly preferably 0.05 to 800 mg/kg (body weight)/day, most preferably 0.1 to 500 mg/kg (body weight)/day. The content of the black rice or an extract thereof may be the amount of intake or administration mentioned above. The amount of intake or administration per day for an adult person through an oral route may be divided appropriately depended on the dosage form to be employed. For example, in the case of capsules, the amount of intake or administration per day for an adult person through an oral route may be divided into 1 to 6 capsules, 1 to 4 capsules, 1 to 3 capsules, or 1 to 2 capsules, per a single dosage.

### [Subject for Application]

When the present invention is used in the field of foods and beverages including functional foods, the subject for application may, for example but not limited to, a person who has an unpleasant clinical condition associated with menstruation. The subject for application may also be a person who is diagnosed as not being affected by dysmenorrhea, a person who does not see a doctor continuously for the diagnosis associated with menstruation, or a person who is not continuously dispensed medication against an unpleasant state caused by menstruation. A subject who does not feel an unpleasant state (discomfort) particularly in a period excluding the menstrual period or the premenstrual period can be determined as an “unaffected person”.

The subject for application may also be, but not limited to, a person in whom organic dysmenorrhea is excluded as the result of a medical interview by a physician or a clinical test. The organic dysmenorrhea can be excluded when it is determined that no abnormality is observed by at least one clinical test selected from the group consisting of an internal examination, an ultrasonographic test, a peripheral blood test, a CRP test, a bacterial culture test, a chlamydia antigen test and a clinical imaging test.

The unpleasant state caused by menstruation tends to be observed in young women most strongly. Therefore, the composition according to the present invention may be a woman in puberty (about 10 to about 18 years old), a women in adolescence (about 15 to about 30 years old), a women of 15 years old or older, or a middle-aged woman (45 years old or older and younger than 55 years old).

The present invention may also include the following aspects.

A composition for ameliorating an unpleasant state caused by menstruation, the composition containing at least one component selected from the group consisting of black rice, cherry blossom, and extracts thereof;
a composition for use for the amelioration of an unpleasant state caused by menstruation, the composition comprising at least one component selected from the group consisting of black rice, cherry blossom, and extracts thereof;
a use of a composition comprising at least one component selected from the group consisting of black rice, cherry blossom, and extracts thereof for the production of an ameliorating agent for an unpleasant state caused by menstruation;
a method for ameliorating an unpleasant state caused by menstruation, the method comprising allowing a composition comprising at least one component selected from the group consisting of black rice, cherry blossom, and extracts thereof to be ingested by a person;
the composition, the use or the method mentioned above, wherein the unpleasant state caused by menstruation is an unpleasant state occurring during the menstrual period or before the menstrual period;
the composition, the use or the method mentioned above, wherein the unpleasant state caused by menstruation is at least one state selected from the group consisting of lower abdominal pain, low back pain, a feeling of abdominal distension, headache, nausea, anemia, diarrhea, tiredness, depression, emotional instability, poor concentration, sleepiness, engorgement of breast (breast tenderness), swelling, rough skin, reduced appetite, decline in efficiency, body weight gain and an incapacitating state;
the composition, the use or the method mentioned above, wherein the unpleasant state occurring before the menstrual period is premenstrual syndrome (PMS) or premenstrual dysphonic disorder (PMDD);
the composition, the use or the method mentioned above, wherein the unpleasant state occurring before the menstrual period is a physical and/or mental unpleasant state occurring 1 to 2 weeks before the menstrual period;
a composition for ameliorating dysmenorrhea, the composition comprising at least one component selected from the group consisting of black rice, cherry blossom, and extracts thereof;
a composition comprising at least one component selected from the group consisting of black rice, cherry blossom, and extracts thereof, the composition being for use in the amelioration of dysmenorrhea;
a use of a composition comprising at least one component selected from the group consisting of black rice, cherry blossom, and extracts thereof for the production of an ameliorating agent for dysmenorrhea;
a method for ameliorating dysmenorrhea, the method comprising allowing a composition comprising at least one component selected from the group consisting of black rice, cherry blossom, and extracts thereof to be ingested by a person;
the composition, the use or the method mentioned above, wherein the dysmenorrhea is functional (primary) dysmenorrhea or organic (secondary) dysmenorrhea;
the composition, the use or the method mentioned above, wherein the black rice is a variety that a purplish black pigment is contained in at least one of a hull or a pericarp of brown rice thereof;
the composition, the use or the method mentioned above, wherein the black rice includes at least one variety selected from the group consisting of “Shikokuen”, “Kurodaen”, “Shonan black rice (registered trade name), “Kokushi” and “kohokushi black rice”;
the composition, the use or the method mentioned above, wherein the black rice extract is an extract of a part of black rice which is selected from the group consisting of a hull, an aleurone layer, a pericarp, a germ and an endosperm;
the composition, the use or the method mentioned above, wherein the polyphenol content in the black rice extract is 3% by mass or more;
the composition, the use or the method mentioned above, wherein the polyphenol content in the black rice extract is 10% by mass or more;
the composition, the use or the method mentioned above, wherein the polyphenol content in the black rice extract is 15% by mass or more;
the composition, the use or the method mentioned above, wherein the cherry blossom is a variety belonging to Subgenus Cerasus (Subgen. Cerasus);
the composition, the use or the method mentioned above, wherein the cherry blossom includes at least one member selected from the group consisting of Japanese cherry (e.g., Prunus cerasus), Prunus avium, Prunus campanulata, Prunus cerasus), Prunus jamasakura, Prunus leveilleana, Prunus pendula, Prunus pseudocerasus, Prunus speciosa, Prunus verecunda, Prunus x mochizukiana, Prunus x yedoensis and Prunus lannesiana;
the composition, the use or the method mentioned above, wherein the cherry blossom includes Prunus lannesiana;
the composition, the use or the method mentioned above, wherein the cherry blossom extract is an extract of at least one part of cherry blossom which is selected from the group consisting of a leaf, a stem, a shaft, a petal, a root and a fruit of cherry blossom;
the composition, the use or the method mentioned above, wherein the caffeoyl glucose content in the cherry blossom extract is 1% by mass or more;
the composition, the use or the method mentioned above, wherein the caffeoyl glucose content in the cherry blossom extract is 1.5% by mass or more;
the composition, the use or the method mentioned above, wherein the caffeoyl glucose content in the cherry blossom extract is 2% by mass or more;
the composition, the use or the method mentioned above, wherein the quercetun glucoside content in the cherry blossom extract is 0.01% by mass or more;
the composition, the use or the method mentioned above, wherein the quercetun glucoside content in the cherry blossom extract is 0.1% by mass or more;
the composition, the use or the method mentioned above, wherein the quercetun glucoside content in the cherry blossom extract is 1% by mass or more;
the composition, the use or the method mentioned above, wherein, in the case of a solid preparation, the content of the at least one component selected from the group consisting of black rice, cherry blossom, and extracts thereof is 0.001 to 95% by mass;
the composition, the use or the method mentioned above, wherein, in the case of a solid preparation, the content of the at least one component selected from the group consisting of black rice, cherry blossom, and extracts thereof is 0.05 to 80% by mass;
the composition, the use or the method mentioned above, wherein, in the case of a liquid preparation, the content of the at least one component selected from the group consisting of black rice, cherry blossom, and extracts thereof is 0.0001 to 25% by mass;
the composition, the use or the method mentioned above, wherein, in the case of a liquid preparation, the content of the at least one component selected from the group consisting of black rice, cherry blossom, and extracts thereof is 0.005 to 10% by mass;
the composition, the use or the method mentioned above, wherein the composition is an oral preparation;
the composition is a solid preparation (e.g., tablets, granules, fine granules, a powder, capsules, chewable tablets) or a liquid preparation (e.g., a drink, a syrup, a suspension);
the composition, the use or the method mentioned above, wherein the amount of ingestion of the at least one component selected from the group consisting of black rice, cherry blossom, and extracts thereof per day for an adult person through an oral route is 0.001 to 2000 mg/kg (body weight)/day;
the composition, the use or the method mentioned above, wherein the amount of ingestion of the at least one component selected from the group consisting of black rice, cherry blossom, and extracts thereof per day for an adult person through an oral route is 0.01 to 1000 mg/kg (body weight)/day;
the composition, the use or the method mentioned above, wherein the amount of ingestion of the at least one component selected from the group consisting of black rice, cherry blossom, and extracts thereof per day for an adult person through an oral route is 0.1 to 500 mg/kg (body weight)/day; and
the composition, the use or the method mentioned above, wherein the subject for application (ingestion) may be a person in puberty (about 10 to about 18 years old) or a person in adolescence (about 15 to about 30 years old), or may be a person of about 15 years old or older or a middle-aged person (about 45 years old and younger than 55 years old).

### EXAMPLES

Next, the present invention will be described specifically by way of examples and test examples. However, the present invention is not limited to the following examples or test examples.

As the black rice extract, black rice extract-P (manufacturer: Oryza Oil & Fat Chemical Co., Ltd.) was used. This extract was a product produced using seeds of black rice (Oryza sativa Linne (Gramineae)) as a raw material and was produced by extracting the seeds with hydrous ethanol. The polyphenol content in the extract was 15% by mass or more.

As the cherry blossom extract, cherry blossom petal extract-P (manufacturer: Oryza Oil & Fat Chemical Co., Ltd.) was used. This extract was a product using petals of Prunus Lannesiana as a raw material and was produced by extracting the petals with hydrous ethanol. The quercetun glucoside content in the extract was 2% by mass or more.

In the following Test Examples, the concentration of the raw material in the extract in each of the above-mentioned commercially available products was employed as a reference value. A powder (0.1 g) of the raw material was suspended and dissolved in PBS (100 μL), and the resultant solution (10 μL) was added to and dissolved in a serum-free DMEM/F12 culture medium (240 μL). The resultant solution (10 μL) was added to and dissolved in an EMT-inducing culture medium mentioned below (190 μL). This solution was used as a 0.2% (W/V) sample, and was diluted 10 folds and 100 folds with the EMT-inducing culture medium to prepare a 0.02 (W/V) % sample and a 0.002 (W/V) % sample, respectively.

### Test Example 1. EMT Inhibition Activity: Focus Formation Inhibition Assay Method

An EMT inhibition activity evaluation method using cells has been studied and established in International Patent Publication No. 2017/104833. In International Patent Publication No. 2017/104833, it is described that, when EMT is induced in retinal pigment epithelial cells (RPE cells), the formation of focuses is observed. An EMT inhibition activity can be evaluated by examining the amount of formed focuses (employing the number and volume of the focuses as measures) depending on the presence or absence of addition of an extract.

A specific evaluation method was as follows.

RPE cells (retinal pigment epithelial cell strain: ARPE-19) were seeded in a plate and were then incubated for 5 days at 37° C. The RPE cells were cultured in an EMT-inducing culture medium supplemented with TNF-α and TGF-β to induce EMT, and simultaneously an extract to be evaluated (an evaluation target extract) was added thereto. As an EMT induction control, a sample without the evaluation target extract was used. Forty-eight hours after the induction of EMT, the solution was treated with a 4% paraformaldehyde solution at room temperature for 30 minutes to fix the cells, and then the resultant sample was washed with PBS three times. The resultant product was treated with a 0.2% Triton-X/PBS solution for 5 minutes, and was then washed with PBS three times. The cells were stained with a fluorescent staining solution (0.2% Phalloidin-Alexa 568 (Molecular Probes, #A12380)/0.05% Hochest 33342 (Invitrogen, #H3570)/3% BSA-PBS) at room temperature for 1 hours, and were then washed with PBS for 5 minutes three times. The fluorescently stained cells were imaged with Image Express Micro (Molecular Devices), and the image was converted to numerical values with MetaXpress 2.0 (Molecular Devices). The amount of formed focuses (focus amount) was analyzed from the numerical data, and an EMT inhibition activity (percentage of inhibition (%)) was calculated in accordance with formula 1 by employing a numerical value obtained when no compound was added as a reference value. In this manner, the EMT inhibition effect in each of the extracts was examined. The amount of formed focuses was determined by employing the number and volume of the focuses as measures. For the purpose of the observation of the whole image under a low magnification, the cells after the acquisition of the fluorescent image were treated with 100% methanol two times at room temperature for 10 minutes, and then a Giemsa stain solution (Nacalai Tesque, Inc., #37114-35) was added to the cells to stain the cells at room temperature for 15 minutes. The cells were washed with methanol three times, and were then dried at room temperature for 3 hours. The dried cells were observed with a microscope.

(EMT inhibition activity)=[(amount of focuses (without extract))−(amount of focuses (with extract))]/(amount of focuses (without extract)×100(%) (Formula 1)

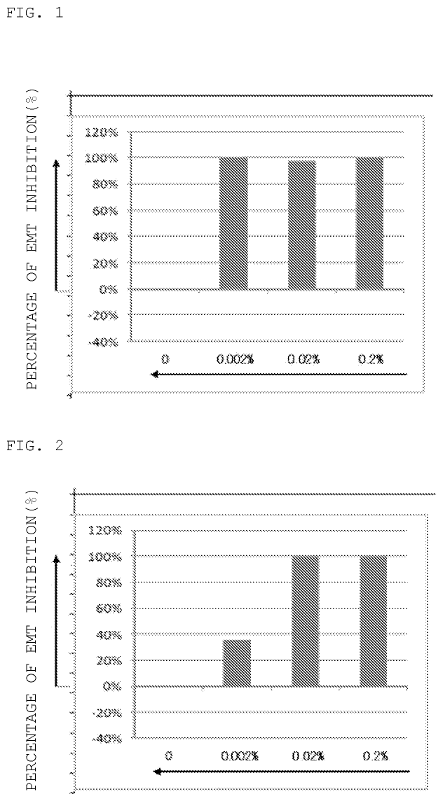

The results of the evaluation of EMT inhibition activities (percentage of inhibition (%)) using a black rice extract are shown in FIG. 1, and the results of the evaluation of EMT inhibition activities (percentage of inhibition (%)) using a cherry blossom petal extract are shown in FIG. 2.

As shown in FIG. 1, with respect to the black rice extract, the EMT inhibition activity was kept at 100% even when the black rice extract was diluted 100-fold (a 0.002% sample).

As shown in FIG. 2, with respect to the cherry blossom petal extract, the EMT inhibition activity was kept at 100% even when the extract stock solution was diluted 10-fold (a 0.02% sample).

As described in Patent Documents 1 and 2, tranilast has an activity to induce the transition of the mesenchymal-like phenotype to the epithelial-like phenotype in an endometrial epithelial cell (i.e., an EMT inhibition activity), and a significant dysmenorrhea-ameliorating effect of tranilast was confirmed both in a patient with functional (primary) dysmenorrhea and a patient with organic (secondary) dysmenorrhea.

Accordingly, it is assumed that the novel component having an anti-EMT activity discovered in the present application can also maintain the transition of phenotype in an endometrial epithelial tissue in a normal state, and therefore can prevent the development of an unpleasant state associated with menstruation and can ameliorate an unpleasant state caused by menstruation.

1. A composition for ameliorating an unpleasant state caused by menstruation, the composition comprising at least one component selected from the group consisting of black rice, cherry blossom and extracts thereof.

2. The composition according to claim 1, wherein the unpleasant state caused by menstruation is an unpleasant state that occurs during a menstrual period or before the menstrual period.

3. The composition according to claim 1, wherein the unpleasant state caused by menstruation is at least one state selected from the group consisting of lower abdominal pain, low back pain, a feeling of abdominal distension, headache, nausea, anemia, diarrhea, tiredness, depression, emotional instability, poor concentration, sleepiness, engorgement of breast (breast tenderness), swelling, rough skin, reduced appetite, decline in efficiency, body weight gain and an incapacitating state.

4. The composition according to claim 1, wherein the black rice extract is an extract of a part of black rice which contains at least one component selected from the group consisting of a hull, an aleurone layer, a pericarp, a germ and an endosperm of black rice.

5. The composition according to claim 1, wherein the cherry blossom extract is an extract of cherry blossom petals.

6. The composition according to claim 1, wherein the composition is an oral preparation.
